(19) United States
(12) Patent Application Publication
(10) Pub. No.: US 2009/0202664 A1
(43) Pub. Date: Aug. 13, 2009

(54) Herbal cream or oil for the treatment hemorroids and wounds

(76) Inventors: Svetislav Mikalacki, Belgrade (YU); Svetlana Mikalachi, Belgrade (YU)

Correspondence Address: Dr. Vera Madzarevic, 34 Barclay Ct, Thornhill, ON, L3T 5T3, CA

(21) Appl. No.: 12/069,410

(22) Filed: Feb. 11, 2008

(51) Int. Cl. A61K 36/38; A61P 17/02; A61P 9/14

(52) U.S. Cl. 424/730

(57) ABSTRACT

This (Patent) protection covers the process for preparing cream for wounds and internal and external hemorrhoids which consists of making herbal-oil extract in three different ways.

Vaseline and lanolin are added to this herbal-oil extract and all is mixed and heated to +40° C. for duration of about 20-30 minutes, when honey is added continuously mixing, up to +35° C., when pollen powder is added with mixing until the mass is homogenized, after which dissolved propolis is added, mixing the mass until it cools of to 25° C. and until the mass is fully homogenized.

To repeat, the herbal-oil extract is made in such way when mix of various herbs in several ways is extracted with ethanol, then mix with olive oil, then alcohol evaporates leaving herbal-oil extract, which is used for preparing medicaments for wounds and hemorrhoids and any other topical use.

### BACKGROUND OF THE INVENTION

Healing with herbs is getting more and more popular and its application will only increase with the time. Actually, synthetic and herbal medicaments are complementing each other and that trend will increase.

This Herbal combination has great advantage over synthetic products because:
They do not have side effects as drugs
The problem with overdosing is not existent, and
They also have multi-functional purpose

There are number of natural medicaments for curing hemorrhoids, and we list only some:
1. Water based extract of chamomile, wild chestnut, holly hock, French rose.
2. Route extract of quince seeds and French rose buds.
3. Extract of Okra (Bendee), symphytum and wide chestnut flower.

Besides these herbal mixes, there are two types of synthetic products, as called anti-hemorrhoids: with and without steroids. Disadvantage of all above products are effectiveness of some products is very low or side effects with synthetic products. Synthetic products cannot be applied on bleeding hemorrhoids therefore healing of bleeding hemorrhoids is not possible. Contrary to this, our product is very successful on bleeding hemorrhoids, which very successfully healed bleeding hemorrhoids.

Technical Problem Definition for Manufacturing Effective Herbal Preparations

With this product 2 problems are solved concurrently: achieved synergetic effect of herbs and homogenization of herbs and insect material (propolis)

Technical Solution

The product is cream for wounds and hemorrhoids, which consist of multi component combination of herb substances, pollen, propolis, and honey with oily base.

This composition of cream allows batch process production, where it is possible mixing and fusion of various substances, cream homogenization, by converting all substances in dissolvable stage.

Hard propolis is converted into dissolvable stage by extraction with ethanol, process that takes 10-15 days at room temperature.

Pollen powder, which is in granules, is dried and grinded into powder.

Honey, which is used for this product, is of black locust or of wild field, since this type of honey is hardest to crystallize.

The suggested process is for preparation of alcohol-herbal extract, which with olive oil transforms into herbal-oil extract, which is used for making cream for wounds and hemorrhoids.

Herbal-oil extract with Vaseline, lanolin, honey, propolis, is therefore used for making cream for wounds and hemorrhoids.

### DETAILED DESCRIPTION OF THE INVENTION

This product is a medicament. Since it consists of herbal material (parts of herbs and pollen, propolis, honey) it belongs to class A 61 K 35/00 of international patent classification.

The products are a cream or oil for wounds and hemorrhoids treatment, which consist of multi component combination of herb substances, pollen, propolis, and honey with oily base.

The composition of the cream allows batch process production, where it is possible mixing and fusion of various substances, cream homogenization, by converting all substances in dissolvable stage. Hard propolis is converted into dissolvable stage by extraction with ethanol, process that takes 10-15 days at room temperature. Pollen powder, which is in granules, is dried and grinded into powder. Honey, which is used for this product, is of black locust or of wild field, since this type of honey is hardest to crystallize. The suggested process is for preparation of alcohol-herbal extract, which with olive oil transforms into herbal-oil extract, which is used for making cream for wounds and hemorrhoids. Herbal-oil extract with Vaseline, lanolin, honey, propolis, is therefore used for making cream for wounds and hemorrhoids.

Process step by step:

Process for Making Herbal-Alcohol Extracts

1. Extract of chestnut (aesculus hippocastanum I.): Cut into very small parts 100 g very dry skin of chestnut, put in utensil, mix it slowly and add 1000 ml of 70% ethanol. All mix well at room temperature several times per day. Extraction takes 15 days. After this period, filter the extract and store it in dark color glass bottle, close the bottle, and use extract when required.

2. Extract of Calendula/Pot marigold (calendula officinalis I.): Put 200 g of fine cut dried Calendula, in utensil, and with mixing add 2000 ml of 70% ethanol. Mix at room temperature several time per day for 15 days. Watch out that temperature never exceeds +40° C. After 15 days filter the extract and store it in dark color glass bottle, close the bottle, and use extract when required.

3. Extract of Boneset comfrey (symphtum officinale I.): Put 200 g of dried and well grounded Boneset comfrey, in utensil, and with mixing add 1000 ml of 70% ethanol. Mix at room temperature several time per day for 15 days. Watch out that temperature never exceeds +40° C. After 15 days filter the extract and store it in dark color glass bottle, close the bottle, and use extract when required. (only used where allowed by regulatory authorities-can be replaced by adding 50% more of chamomile extract)

4. Herbal extract of Yarrow (Milfoil) (achillea millefolium I.): Put 100 g of dried and well crushed Yarrow in utensil, and with mixing add 1000 ml of 70% ethanol. Mix at room temperature several time per day for 15 days. Watch out that temperature never exceeds +40° C. After 15 days filter the extract and store it in dark color glass bottle, close the bottle, and use extract when required.

5. Herbal extract of Chamomile (matricaria chamomillam I.): Put 100 g of dried and well crushed Chamomile flower in utensil, and with mixing seep in slowly 1000 ml of 70% ethanol. Mix at room temperature several time per day for 15 days. Watch out that temperature never exceeds +40° C. After 15 days filter the extract and store it in dark color glass bottle, close the bottle, and use extract when required.

6. Herbal extract of Shepherd's purse/Pick pocket (capsella bursa pastoris I.): Put 100 g of dried and well crushed whole Shepherd's purse in utensil, and with mixing seep in slowly 1000 ml of 70% ethanol. Mix at room temperature several time per day for 15 days. Watch out that temperature never exceeds +40° C. After 15 days filter the extract and store it in dark color glass bottle, close the bottle, and use extract when required.

7. Herbal extract of Common bistort (Adderwort) (gentiana lutea I.): Put 100 g of dried and well crushed root of Common bistort in utensil, and with mixing add 1000 ml of 70% ethanol. Mix at room temperature several time per day for 15 days. Watch out that temperature never exceeds +40° C. After 15 days filter the extract and store it in dark color glass bottle, close the bottle, and use extract when required.

8. Herbal extract of St Johns Worth (hypericum perforatum I.): Put 100 g of dried and well grounded herbs of St Johns worth in utensil, and with mixing add 1000 ml of 70% ethanol. Mix at room temperature several time per day for 15 days. Watch out that temperature never exceeds +40° C. After 15 days filter the extract and store it in dark color glass bottle, close the bottle, and use extract when required.

Process of Making Herbal-Oil Extract (“Olea Infuzi”)

Herbal-oil extract is made in the following way: 93.3 g of dry, fine crashed aesculus hyppocastanum I. mix with 116.6 g of calendula officinalis I., 70 g achillea millefolium I., 140 g matricaria chamomilla I., 70 g of capsella bursa pastoris I., 70 g of gentiana lutea I, and 93.3 g hypericum perforatum I., all in one utensil and pour over it with 650 ml of 96% ethanol, mix well, cover and leave for minimum 18 hours.

After that time, add 6530 ml of pure olive oil and start heating up with continuously mixing at 70-90° C. temperature. During that time alcohol will start evaporating. This will last several hours. After evaporation of alcohol (detected by smell), filter the mix and settlement of the herbs on the bottom of the utensil is washed with about 60° C. worm pure olive oil. Filter that residual and mix both filtered residuals and leave for 18 hours for herb remaining to settle.

Clear herbal extract is separated and remaining mass is filtered again. At this point about 6530 ml of herbal-oil extract—active component is made.

This extract is now ready for making cream for hemorrhoids.

18 g of this herbal-oil extract is mixed with 48.96 g Vaseline and 12.9 g lanoline and such mix is heated to 40° C. during 20-30 minutes, until mixture is fully dissolved and homogenized. In this worm mixture, with continuous mixing add 49.9 g honey. Decrease temperature to 35° C. and while mixing, add 6.93 g pollen dust. Mix until mass is fully homogenized. Add 10.26 ml of dissolved propolis (propolis dissolved in 96% ethanol). Continue mixing until mass is homogenized and decrease temperature gradually to 25° C.

After that the cream for hemorrhoids is ready for packaging.

Laboratory Process for Making Herbal-Oil Extract “Hemorom”

In 2 L glass jar with three openings, equipped with mixer, vertical condenser, thermometer, heater put: 20 g of wild chestnut skin, 25 g Pot marigold (Calendula) flower, 15 g of Common comfrey root, 15 g Yarrow flower and leaf, 15 g Chamomile flower, 15 g Shepard's purse (part of herb above ground), 15 g Yellow gentian root, and 20 g ST. Johns worth.

In the above add 70-80 ml of 96% ethanol and with continuous mixing seep slowly 1400 ml of pure olive oil and start heating to +50 C. heating takes 30 hours, when vertical condenser is replaced with Libig condenser and when temperature drops to 30 C, under vacuum to extract alcohol, add 280 ml fresh olive oil, replace Libig condenser with the vertical condenser, heat up the mix to +50 C with continuous mixing. After 20-30 minutes filter still worm mixture. Leave this extract for minimum 24 hours for herbal particles to settle down until oil becomes clear. Upper part of extract is removed and reminder slurry is filtered.

The final herbal extract is yellow clear color of about 1436 ml or about 85.5% of added olive oil. We are of opinion that olive oil waste will be reduced in industrial production of this extract.

Solid portion of herbs, after separation from oil is not used but could be burnt as a fuel.

Preparation of Herbal-Alcohol Extract

In utensil mix dry well crushed herbs: 93.3 g of chestnut, 116.6 g Pot marigold (Calendula), 70 g of Common comfrey, 70 g Yarrow, 70 g Chamomile, 70 g Shepard's purse, 70 g Common bistort, and 93.3 g ST. Johns worth. Mixing slowly seep 6532 ml of 70% ethanol. Mix at room temperature several time per day for 15 days. Watch out that temperature never exceeds +40° C. After 15 days filter the extract and reminder sludge is washed by 1300 ml of 70% ethanol and both extracts are mixed and stored in dark color glass bottle. About 6,500 ml of herbal-alcohol extracts is made this way.

In 6,000 ml of virgin olive oil mix slowly 6,000 ml above herbal-alcohol extract (aesculus hippocastanum I., calendula officinalis I., achillea millefolium I., matricaria chamomilla I., capsella bursa pastoris I., gentiana lutea I., hypericum prforatum I.).

Heat slowly this mix at 70-90° C. evaporating alcohol. Extraction in oil is taking place for about 5-6 hours. After evaporation of alcohol (detected by smell), filter the extract.

Such made oil extract (which is now active component) is ready for further use.

The extract is stored it in dark color glass bottle, close the bottle, and use extract when required.

Process for Making Herbs Oils Extract

In 1,400 ml of virgin olive oil seep slowly with continuous mixing 200 ml of herbal-alcohol extract of chestnut (aesculus hippocastanum I.), 250 ml of herbal-alcohol extract of Calendula/Pot marigold (calendula officinalis I.), 150 ml of herbal-alcohol extract of Boneset comfrey (symphytum officinale I.), 150 ml of herbal-alcohol extract of Yarrow/Milfoil (achillea millefolium I.), 150 ml of herbal-alcohol extract of Chamomile (matricaria chamomillam I.), 150 ml of herbal-alcohol extract of Shepherd's purse/Pick pocket (capsella bursa pastoris I.), 150 ml of herbal-alcohol extract of Common bistort/Adderwort (gentiana lutea I.), and 200 ml of herbal-alcohol extract of St. Johns wort (hypericum perforatum I).

Heat slowly this mix at 70-90° C. evaporating alcohol. Extraction in oil is taking place for about 2.5-3 hours. After evaporation of alcohol (detected by smell), filter the extract.

With this process about 1,400 ml of oil extract is generated, which is ready for further processing.

The extract is stored it in dark color glass bottle, close the bottle, and use extract when required.

1. A Composition comprising Extract of chestnut (aesculus hippocastanum I.), Extract of Calendula/Pot marigold (calendula officinalis I.). Extract of Boneset comfrey (symphtum officinale I.). Herbal extract of Yarrow (Milfoil) (achillea millefolium I.). Herbal extract of Chamomile (matricaria chamomillam I.). Herbal extract of Shepherd's purse/Pick pocket (capsella bursa pastoris I.). Herbal extract of Common bistort (Adderwort) (gentiana lutea I.). Herbal extract of St Johns Worth (hypericum perforatum I.)

2. A Process for the manufacturing of HERBAL-ALCOHOL EXTRACTS in claim 1

3. A Process for the Manufacturing of HERBAL-OIL EXTRACT (“OLEA INFUZI”)

4. A LABORATORY PROCESS FOR MAKING HERBAL-OIL EXTRACT “HEMOROM”

5. A METHOD OF PREPARATION OF HERBAL-ALCOHOL EXTRACT with the Extracts in claim 1

6. A PROCESS FOR MAKING HERBS OILS EXTRACT with the Extracts in claim 1

7. A method for the treatment of hemorrhoids, wounds, scars, veins, and all other potential topical applications in mammals utilizing products in claim 1, 3, 4, 5 and 6.

8. A Method of claim 7 where in mammal is a human

9. A method for alleviating symptoms associated with hemorrhoids comprising administering a therapeutically effective amount of the composition of claim 1, 3, 4, 5 and 6 to a mammal.
